(19) United States
(12) Patent Application Publication
(10) Pub. No.: US 2004/0009980 A1
(43) Pub. Date: Jan. 15, 2004

(54) Calcilytic compounds

(76) Inventors: Pradip Bhatnagar, King of Prussia, PA (US); Joelle L. Burgess, Collegeville, PA (US); James F. Callahan, Collegeville, PA (US); Maria A. Lago, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/415,120

(22) PCT Filed: Oct. 25, 2001
(86) PCT No.: PCT/US01/46184

(51) Int. Cl. A61K 31/53; A61K 31/513; A61K 31/50; A61K 31/4965; A61K 31/44; C07D 213/63

(52) U.S. Cl. 514/241; 514/247; 514/252.01; 514/252.1; 514/269; 514/345; 546/290; 544/182; 544/239; 544/316

(57) ABSTRACT

Novel phosphate esters compounds and methods of using them as calcilytic compounds are provided.

### FIELD OF INVENTION

The present invention relates to novel calcilytic compounds, pharmaceutical compositions containing these compounds and their use as calcium receptor antagonists.

In mammals, extracellular Ca²⁺ is under rigid homeostatic control and regulates various processes such as blood clotting, nerve and muscle excitability, and proper bone formation. Extracellular Ca²⁺ inhibits the secretion of parathyroid hormone (“PTH”) from parathyroid cells, inhibits bone resorption by osteoclasts, and stimulates secretion of calcitonin from C-cells. Calcium receptor proteins enable certain specialized cells to respond to changes in extracellular Ca²⁺ concentration.

PTH is the principal endocrine factor regulating Ca²⁺ homeostasis in the blood and extracellular fluids. PTH, by acting on bone and kidney cells, increases the level of Ca²⁺ in the blood. This increase in extracellular Ca²⁺ then acts as a negative feedback signal, depressing PTH secretion. The reciprocal relationship between extracellular Ca²⁺ and PTH secretion forms an important mechanism maintaining bodily Ca²⁺ homeostasis.

Extracellular Ca²⁺ acts directly on parathyroid cells to regulate PTH secretion. The existence of a parathyroid cell surface protein which detects changes in extracellular Ca²⁺ has been confirmed. See Brown et al., Nature 366:574, 1993. In parathyroid cells, this protein, the calcium receptor, acts as a receptor for extracellular Ca²⁺, detects changes in the ion concentration of extracellular Ca²⁺, and initiates a functional cellular response, PTH secretion.

Extracellular Ca²⁺ influences various cell functions, reviewed in Nemeth et al., Cell Calcium 11:319, 1990. For example, extracellular Ca²⁺ plays a role in parafollicular (C-cells) and parathyroid cells. See Nemeth, Cell Calcium 11:323, 1990. The role of extracellular Ca²⁺ on bone osteoclasts has also been studied. See Zaidi, Bioscience Reports 10:493, 1990.

Various compounds are known to mimic the effects of extra-cellular Ca²⁺ on a calcium receptor molecule. Calcilytics are compounds able to inhibit calcium receptor activity, thereby causing a decrease in one or more calcium receptor activities evolved by extracellular Ca²⁺. Calcilytics are useful as lead molecules in the discovery, development, design, modification and/or construction of useful calcium modulators, which are active at Ca²⁺ receptors. Such calcilytics are useful in the treatment of various disease states characterized by abnormal levels of one or more components, e.g., polypeptides such as hormones, enzymes or growth factors, the expression and/or secretion of which is regulated or affected by activity at one or more Ca²⁺ receptors. Target diseases or disorders for calcilytic compounds include diseases involving abnormal bone and mineral homeostasis.

Abnormal calcium homeostasis is characterized by one or more of the following activities: an abnormal increase or decrease in serum calcium; an abnormal increase or decrease in urinary excretion of calcium; an abnormal increase or decrease in bone calcium levels (for example, as assessed by bone mineral density measurements); an abnormal absorption of dietary calcium; an abnormal increase or decrease in the production and/or release of messengers which affect serum calcium levels such as PTH and calcitonin; and an abnormal change in the response elicited by messengers which affect serum calcium levels.

Thus, calcium receptor antagonists offer a unique approach towards the pharmacotherapy of diseases associated with abnormal bone or mineral homeostasis, such as hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

### SUMMARY OF THE INVENTION

The present invention comprises novel calcium receptor antagonists represented by Formula (I) hereinbelow and their use as calcium receptor antagonists in the treatment of a variety of diseases associated with abnormal bone or mineral homeostasis, including but not limited to hypoparathyroidism, osteosarcoma, periodontal disease, fracture healing, osteoarthritis, rheumatoid arthritis, Paget's disease, humoral hypercalcemia associated with malignancy and fracture healing, and osteoporosis.

The present invention further provides a method for antagonizing calcium receptors in an animal, including humans, which comprises administering to an animal in need thereof an effective amount of a compound of Formula (I), indicated hereinbelow.

The present invention further provides a method for increasing serum parathyroid levels in an animal, including humans, which comprises administering to an animal in need thereof an effective amount of a compound of Formula (I), indicated herein below.

### DETAILED DESCRIPTION OF THE INVENTION

The compounds of the present invention are selected from Formula (I) herein below:

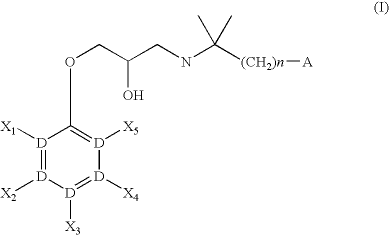

wherein:

A is an aryl or fused aryl, dihydro or tetrahydro fused aryl, heteroaryl or fused heteroaryl, dihydro or tetrahydro fused heteroaryl, unsubstituted or substituted with any substituent being selected from the group consisting of OH, halogen, C₁₋₄alkyl, C₁₋₄alkoxy, C₃₋₆ cycloalkyl, CF₃, OCF₃, CN, and NO₂;

D is C or N with 1-2-N in ring provided that X₁-X₅ are not present when D is N;

X₁ and X₅ are, independently, selected from the group consisting of H, halogen, CN, and NO₂, provided that either X₁ or X₅ is H; further provided that X₁ and X₅ are not present when D is N;

X₂, X₃ and X₄ are selected from the group consisting of H, halogen, O—C₁₋₄ alkyl, and J-K, wherein:

J is a covalent bond, alkylene, O-alkylene or alkenylene; and

K is selected from the group consisting of, CO₂R₅, CONR₄R′₄, OH, NR₄R′₄ and CN and provided X₂, X₃ and X₄ are not present when D is N;

R₄ and R′₄ are independently H, alkyl, aryl or heteroaryl;

R₅ is H, alkyl, alkyl-(O-alkyl)ₘ-O-alkyl, aryl or heteroaryl;

n is an integer from 0 to 4; and,

m is an integer from 1-3.

As used herein, “alkyl” refers to an optionally substituted hydrocarbon group joined by single carbon-carbon bonds and having 1-20 carbon atoms joined together. The alkyl hydrocarbon group may be linear, branched or cyclic, saturated or unsaturated. Preferably, substituents on optionally substituted alkyl are selected from the group consisting of aryl, CO₂R, CO₂NHR, OH, OR, CO, NH₂, halo, CF₃, OCF₃ and NO₂, wherein R represents H, C 1-4 alkyl, C₃₋₆ cycloalkyl, C₂₋₅ alkenyl, C₂₋₅ alkynyl, heterocycloalkyl, or aryl. Additional substituents are selected from F, Cl, Br, I, N, S and O. Preferably, no more than three substituents are present. More preferably, the alkyl has 1-12 carbon atoms and is unsubstituted. Preferably, the alkyl group is linear.

As used herein “cycloalkyl” refers to optionally substituted 3-7 membered carbocyclic rings wherein any substituents are selected from the group consisting of, F, Cl, Br, I, N(R₄)₂, SR₄ and OR₄, unless otherwise indicated.

As used herein, “aryl” refers to an optionally substituted aromatic group with at least one ring having a conjugated pi-electron system, containing up to two conjugated or fused ring systems. Aryl includes carbocyclic aryl, and biaryl groups, all of which may be optionally substituted. Preferred aryl include phenyl and naphthyl. More preferred aryl include phenyl. Preferred substituents are selected from the group consisting of halogen, C₁₋₄ alkyl, OCF₃, CF₃, OMe, CN, OSO₂ R and NO_{2,} wherein R represents C₁₋₄ alkyl or C₃-6 cycloalkyl.

As used herein, “heteroaryl” refers to an aryl ring containing 1,2 or 3 heteroatoms such as N, S, or O.

As used herein, “alkenyl” refers to an optionally substituted hydrocarbon group containing at least one carbon-carbon double bond and containing up to 5 carbon atoms joined together. The alkenyl hydrocarbon chain may be straight, branched or cyclic. Any substituents are selected from the group consisting of halogen, C₁₋₄ alkyl, OCF₃, CF₃, OMe, CN, OSO₂ R and NO₂, wherein R represents C₁₋₄ alkyl or C₃₋₆ cycloalkyl.

As used herein, “alkynyl” refers to an optionally substituted hydrocarbon group containing at least one carbon-carbon triple bond between the carbon atoms and containing up to 5 carbon atoms joined together. The alkynyl hydrocarbon group may be straight-chained, branched or cyclic. Any substituents are selected from the group consisting of halogen, C₁₋₄ alkyl, OCF₃, CF₃, OMe, CN, OSO₂ R and NO₂, wherein R represents

C₁₋₄ alkyl or C₃₋₆ cycloalkyl.

The compounds of the present invention may contain one or more asymmetric carbon atoms and may exist in racemic and optically active forms. All of these compounds and diastereomers are contemplated to be within the scope of the present invention.

Preferred compounds of the present inventions include:

3-{6-Cyano-5-[(R)-2-hydroxy-3-(2-indan-2-yl-1,1-dimethyl-ethylamino)-propoxy]-pyridin-2-yl}-propionic acid ethyl ester;

3-{6-Cyano-5-[(R)-2-hydroxy-3-(2-indan-2-yl-1,1-dimethyl-ethylamino)-propoxy]-pyridin-2-yl}-propionic acid;

3-(6-Cyano-5-{(R)-2-hydroxy-3-[2-(4-methoxy-phenyl)-1,1-dimethyl-ethylamino]-propoxy}-pyridin-2-yl)-propionic acid ethyl ester;

3-(6-Cyano-5-{(R)-2-hydroxy-3-[2-(4-methoxy-phenyl)-1,1-dimethyl-ethylamino]-propoxy}-pyridin-2-yl)-propionic acid;

3-(6-Cyano-5-{(R)-2-hydroxy-3-[4-(2-methoxy-phenyl)-1,1-dimethyl-butylamino]-propoxy}-pyridin-2-yl)-propionic acid ethyl ester; and

3-(6-Cyano-5-{(R)-2-hydroxy-3-[4-(2-methoxy-phenyl)-1,1-dimethyl-butylamino]-propoxy}-pyridin-2-yl)-propionic acid.

Pharmaceutically acceptable salts are non-toxic salts in the amounts and concentrations at which they are administered.

Pharmaceutically acceptable salts include acid addition salts such as those containing sulfate, hydrochloride, fumarate, maleate, phosphate, sulfamate, acetate, citrate, lactate, tartrate, methanesulfonate, ethanesulfonate, benzenesulfonate, p-toluenesulfonate, cyclohexylsulfamate and quinate. A preferred salt is a hydrochloride. Pharmaceutically acceptable salts can be obtained from acids such as hydrochloric acid, maleic acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, fumaric acid, and quinic acid.

Pharmaceutically acceptable salts also include basic addition salts such as those containing benzathine, chloroprocaine, choline, diethanolamine, ethylenediamine, meglumine, procaine, aluminum, calcium, lithium, magnesium, potassium, sodium, ammonium, alkylamine, and zinc, when acidic functional groups, such as carboxylic acid or phenol are present.

The present invention provides compounds of Formula (I) above, which can be prepared using standard techniques. An overall strategy for preparing preferred compounds described herein can be carried out as described in this section. The examples, which follow, illustrate the synthesis of specific compounds. Using the protocols described herein as a model, one of ordinary skill in the art can readily produce other compounds of the present invention.

All reagents and solvents were obtained from commercial vendors. Starting materials were synthesized using standard techniques and procedures.

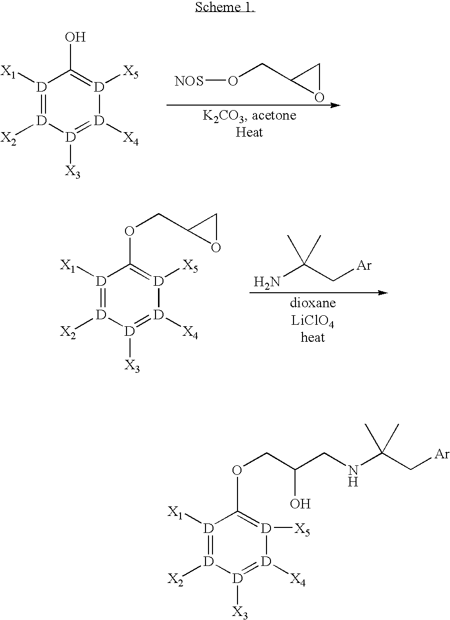

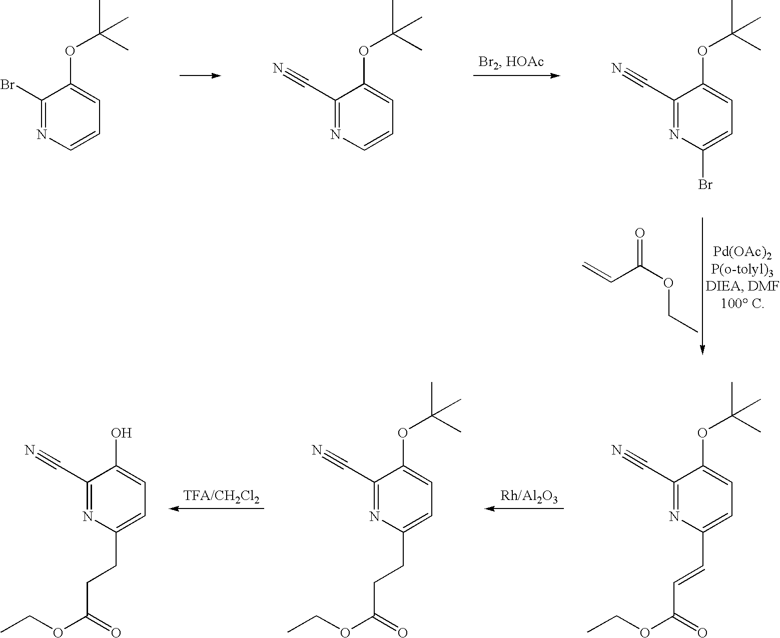

General Preparation

A general procedure used to synthesize many of the compounds can be carried out as described in Scheme 1, above: A solution of heteroaryl alcohol in acetone was treated with an appropriate base such as K₂CO₃, heated for 15 min. R-glycidyl nosylate was added and the reaction continued overnight to give the corresponding glycidyl ether (Scheme 1). A solution of the substituted glycidyl ether and excess amine (e.g., 1,1-dimethyl-2-(4-methyloxyphenyl)ethylamine) in absolute ethanol, acetonitrile, THF, dioxane, toluene or any other similar solvent in the presence of a suitable catalyst such as LiClO₄ is stirred overnight at reflux. The product is purified by chromatography. Hydrochloride salts are prepared by treatment of the corresponding free base with HCl either in gas phase or 4M dioxane solution, or any other standard method. A representative method to prepare the heteroaryl alcohol is shown in Scheme 2. 2-Bromo-pyridin-3-ol is treated with isobutylene in CH₂Cl₂ in the presence of H₂SO₄ to give the corresponding t-butyl ester, which is treated with Zn(CN)₂ with Pd catalysis to give 3-tert-butoxy-pyridine-2-carbonitrile. Selective bromination gives 6-bromo-3-tert butoxy-pyridine-2-carbonitrile. Heck coupling with ethyl acrylate followed by selective reduction gives 3-(5-tert-butoxy-6-cyano-pyridin-2-yl)-propionic acid ethyl ester which is deprotected with TFA to give the 3-(5-hydroxy-6-cyano-pyridin-2-yl)-propionic acid ethyl ester.

In order to use a compound of Formula (I) or a pharmaceutically acceptable salt thereof for the treatment of humans and other mammals, it is normally formulated in accordance with standard pharmaceutical practice as a pharmaceutical composition.

The calcilytic compounds can be administered by different routes including intravenous, intraperitoneal, subcutaneous, intramuscular, oral, topical (transdermal), or transmucosal administration. For systemic administration, oral administration is preferred. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g., intramuscular, intravenous, intraperitoneal, and subcutaneous. For injection, the compounds of the invention are formulated in liquid solutions, preferably, in physiologically compatible buffers or solutions, such as saline solution, Hank's solution, or Ringer's solution. In addition, the compounds may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the compounds of the invention can be formulated into ointments, salves, gels, or creams, as is generally known in the art.

The amounts of various calcilytic compounds to be administered can be determined by standard procedures taking into account factors such as the compound IC₅₀, EC₅₀, the biological half-life of the compound, the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (I) or a pharmaceutically acceptable salt thereof, calculated as the free base. The daily dosage for parenteral, nasal, oral inhalation, transmucosal or transdermal routes contains suitably from 0.01 mg to 100 mg/Kg, of a compound of Formula (I). A topical formulation contains suitably 0.01 to 5.0% of a compound of Formula (I). The active ingredient may be administered, for example, from 1 to 6 times per day, preferably once, sufficient to exhibit the desired activity, as is readily apparent to one skilled in the art.

As used herein, “treatment” of a disease includes, but is not limited to prevention, retardation and prophylaxis of the disease.

Diseases and disorders which might be treated or prevented, based upon the affected cells, include bone and mineral-related diseases or disorders; hypoparathyroidism; those of the central nervous system such as seizures, stroke, head trauma, spinal cord injury, hypoxia-induced nerve cell damage, such as occurs in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome; diseases involving excess water reabsorption by the kidney, such as syndrome of inappropriate ADH secretion (SIADH), cirrhosis, congestive heart failure, and nephrosis; hypertension; preventing and/or decreasing renal toxicity from cationic antibiotics (e.g., aminoglycoside antibiotics); gut motility disorders such as diarrhea and spastic colon; GI ulcer diseases; GI diseases with excessive calcium absorption such as sarcoidosis; autoimmune diseases and organ transplant rejection; squamous cell carcinoma; and pancreatitis.

In a preferred embodiment of the present invention, the present compounds are used to increase serum parathyroid hormone (“PTH”) levels. Increasing serum PTH levels can be helpful in treating diseases such as hypoparathyroidism, osteosarcoma, periodontal disease, fracture, osteoarthritis, rheumatoid arthritis, Paget's disease, humoral hypercalcemia malignancy and osteoporosis.

In a preferred embodiment of the present invention, the present compounds are co-administered with an anti-resorptive agent. Such agents include, but are not limited estrogen, 1, 25 (OH)₂ vitamin D3, calcitonin, selective estrogen receptor modulators, vitronectin receptor antagonists, V−H+-ATPase inhibitors, src SH2 antagonists, bisphosphonates and cathepsin K inhibitors.

Another aspect of the present invention describes a method of treating a patient comprising administering to the patient an amount of a present compound sufficient to increase the serum PTH level. Preferably, the method is carried out by administering an amount of the compound effective to cause an increase in duration and/or quantity of serum PTH level sufficient to have a therapeutic effect.

In various embodiments, the compound administered to a patient causes an increase in serum PTH having a duration of up to one hour, about one to about twenty-four hours, about one to about twelve hours, about one to about six hours, about one to about five hours, about one to about four hours, about two to about five hours, about two to about four hours, or about three to about six hours.

In an alternative embodiment of the present invention, the compound administered to a patient causes an increase in serum PTH having a duration of more than about twenty four hours provided that it is co-administered with an anti resorptive agent.

In additional different embodiments, the compound administered to a patient causes an increase in serum PTH of up to two fold, two to five fold, five to ten fold, and at least 10 fold, greater than peak serum PTH in the patient. The peak serum level is measured with respect to a patient not undergoing treatment.

Composition of Formula (I) and their pharmaceutically acceptable salts, which are active when given orally, can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane.

A typical suppository formulation comprises a compound of Formula (I) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

Preferably the composition is in unit dosage form, for example a tablet, capsule or metered aerosol dose, so that the patient may administer a single dose.

No unacceptable toxological effects are expected when compounds of the present invention are administered in accordance with the present invention.

The biological activity of the compounds of Formula (I) are demonstrated by the following tests:

(I) Calcium Receptor Inhibitor Assay

Calcilytic activity was measured by determining the IC₅₀ of the test compound for blocking increases of intracellular Ca²⁺ elicited by extracellular Ca²⁺ in HEK 293 4.0-7 cells stably expressing the human calcium receptor. HEK 293 4.0-7 cells were constructed as described by Rogers et al., J. Bone Miner. Res. 10 Suppl. 1:S483, 1995 (hereby incorporated by reference herein). Intracellular Ca²⁺ increases were elicited by increasing extracellular Ca²⁺ from 1 to 1.75 mM. Intracellular Ca²⁺ was measured using fluo-3, a fluorescent calcium indicator.

The procedure was as follows:

1. Cells were maintained in T-150 flasks in selection media (DMEM supplemented with 10% fetal bovine serum and 200 ug/mL hygromycin B), under 5% CO₂:95% air at 37° C. and were grown up to 90% confluency.

2. The medium was decanted and the cell monolayer was washed twice with phosphate-buffered saline (PBS) kept at 37° C. After the second wash, 6 mL of 0.02% EDTA in PBS was added and incubated for 4 minutes at 37° C. Following the incubation, cells were dispersed by gentle agitation.

3. Cells from 2 or 3 flasks were pooled and pelleted (100×g). The cellular pellet was resuspended in 10-15 mL of SPF-PCB+ and pelleted again by centrifugation. This washing was done twice.

Sulfate- and phosphate-free parathyroid cell buffer (SPF-PCB) contains 20 mM Na-Hepes, pH 7.4, 126 mM NaCl, 5 mM KCl, and 1 mM MgCl₂. SPF-PCB was made up and stored at 4° C. On the day of use, SPF-PCB was supplemented with 1 mg/mL of D-glucose and 1 mM CaCl₂ and then split into two fractions. To one fraction, bovine serum albumin (BSA; fraction V, ICN) was added at 5 mg/mL (SPF−PCB+). This buffer was used for washing, loading and maintaining the cells. The BSA-free fraction was used for diluting the cells in the cuvette for measurements of fluorescence.

4. The pellet was resuspended in 10 mL of SPF-PCB+containing 2.2 uM fluo-3 (Molecular Probes) and incubated at room temperature for 35 minutes.

5. Following the incubation period, the cells were pelleted by centrifugation. The resulting pellet was washed with SPF-PCB+. After this washing, cells were resuspended in SPF-PCB+at a density of 1-2×10⁶ cells/mL.

6. For recording fluorescent signals, 300 uL of cell suspension were diluted in 1.2 mL of SPF buffer containing 1 mM CaCl₂ and 1 mg/mL of D-glucose. Measurements of fluorescence were performed at 37° C. with constant stirring using a spectrofluorimeter. Excitation and emission wavelengths were measured at 485 and 535 nm, respectively. To calibrate fluorescence signals, digitonin (5 mg/mL in ethanol) was added to obtain Fmax, and the apparent Fmin was determined by adding Tris-EGTA (2.5 M Tris-Base, 0.3 M EGTA). The concentration of intracellular calcium was calculated using the following equation:

Intracellular calcium=(F−Fₘᵢₙ/Fₘₐₓ)×K_{d}; where K_{d}=400 nM.

7. To determine the potential calcilytic activity of test compounds, cells were incubated with test compound (or vehicle as a control) for 90 seconds before increasing the concentration of extracellular Ca²⁺ from 1 to 2 mM. Calcilytic compounds were detected by their ability to block, in a concentration-dependent manner, increases in the concentration of intracellular Ca²⁺ elicited by extracellular Ca²⁺.

In general, those compounds having lower IC₅₀ values in the Calcium Receptor Inhibitor Assay are more preferred compounds. Compounds having an IC₅₀ greater than 50 uM were considered to be inactive. Preferred compounds are those having an IC₅₀ of 10 uM or lower, more preferred compounds have an IC₅₀ of 1 uM, and most preferred compounds have an IC₅₀ of 0.1 uM or lower.

(II) Calcium Receptor Binding Assay

HEK 293 4.0-7 cells stably transfected with the Human Parathyroid Calcium Receptor (“HuPCaR”) were scaled up in T180 tissue culture flasks. Plasma membrane is obtained by polytron homogenization or glass douncing in buffer (50 mM Tris-HCl pH 7.4, 1 mM EDTA, 3 mM MgCl₂) in the presence of a protease inhibitor cocktail containing 1 uM Leupeptin, 0.04 uM Pepstatin, and 1 mM PMSF. Aliquoted membrane was snap frozen and stored at −80° C. ³H labeled compound was radiolabeled to a radiospecific activity of 44 Ci/mmole and was aliquoted and stored in liquid nitrogen for radiochemical stability.

A typical reaction mixture contains 2 nM ³H compound ((R,R)-N4′-Methoxy-t-3-3′-methyl-1′-ethylphenyl-1-(1-naphthyl)ethylamine), or ³H compound (R)-N-[2-Hydroxy-3-(3-chloro-2-cyanophenoxy)propyl]-1,1-dimethyl-2-(4-methoxyphenyl)ethylamine 4-10 ug membrane in homogenization buffer containing 0.1% gelatin and 10% EtOH in a reaction volume of 0.5 mL. Incubation is performed in 12×75 polyethylene tubes in an ice water bath. To each tube 25 uL of test sample in 100% EtOH is added, followed by 400 uL of cold incubation buffer, and 25 uL of 40 nM ³H-compound in 100% EtOH for a final concentration of 2 nM. The binding reaction is initiated by the addition of 50 uL of 80-200 ug/mL HEK 293 4.0-7 membrane diluted in incubation buffer, and allowed to incubate at 4° C. for 30 min. Wash buffer is 50 mM Tris-HCl containing 0.1% PEI. Nonspecific binding is determined by the addition of 100-fold excess of unlabeled homologous ligand, and is generally 20% of total binding. The binding reaction is terminated by rapid filtration onto 1% PEI pretreated GF/C filters using a Brandel Harvestor. Filters are placed in scintillation fluid and radioactivity assessed by liquid scintillation counting.

### EXAMPLES

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (*) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Continuous wave infrared (IR) spectra were recorded on a Perkin-Elmer 683 infrared spectrometer, and Fourier transform infrared (FTIR) spectra were recorded on a Nicolet Impact 400 D infrared spectrometer. IR and FTIR spectra were recorded in transmission mode, and band positions are reported in inverse wavenumbers (cm⁻¹). Mass spectra were taken on either VG 70 FE, PE Syx API III, or VG ZAB HF instruments, using fast atom bombardment (FAB) or electrospray (ES) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatographs were carried out on E. Merck Kieselgel 60 (230-400-mesh) silica gel. Analytical and preparative HPLC were carried out on Rainin or Gilson chromatographs. ODS refers to an octadecylsilyl derivatized silica gel chromatographic support. 5μ Apex-ODS indicates an octadecylsilyl derivatized silica gel chromatographic support having a nominal particle size of 5μ, made by Jones Chromatography, Littleton, Colo. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev.) Celite® is a filter aid composed of acid-washed diatomaceous silica, and is a registered trademark of Manville Corp., Denver, Colo. Following the general procedure described above the following compounds have been synthesized: Example 1

Preparation of 3-(5-Hydroxy-6-cyano-pyridin-2-yl)-propionic Acid Ethyl Ester

a) 2-Bromo-3-tert-butoxy-pyridine

2-Bromo-pyridin-3-ol is treated with isobutylene in CH₂Cl₂ in the presence of H₂SO₄ at −78° C. and then stirred at reflux for 18 h. The reaction mixture is evaporated and the residue in ethyl acetate is washed with 5% Na₂CO₃ (aqueous), dried and evaporated to give the above titled compound.

b) 3-tert-Butoxy-pyridine-2-carbonitrile

Using the method of Maligres, et al. (Tetrahedron Lett 40, 8193, 2000), the compound from Example 1(a) in wet DMF is treated with Zn(CN)₂, Pd₂(dba)₃ and DPPF at 120° C. for 20 h. The reaction mixture is evaporated and the residue in ethyl acetate is washed with 5% Na₂CO₃ (aqueous), dried and evaporated. Purification by flash chromatography gives the above titled compound.

c) 6-Bromo-3-tert-butoxy-pyridine-2-carbonitrile

The compound from Example 1(b) in acetic acid is treated with 1 equivalent of Br₂ at 0° C. After all of the starting material is consumed the reaction is quenched by the addition of sodium thiosulfate. The reaction is evaporated and the residue in ethyl acetate is washed with 5% Na₂CO₃ (aqueous), dried and evaporated. Purification by flash chromatography gives the above titled compound.

d) 3-(5-tert-Butoxy-6-cyano-pyridin-2-yl)-acrylic acid ethyl ester

A mixture of compound from Example 1 (c), ethyl acrylate, Pd(OAc)₂, P(o-tolyl)3, and DIEA in DMF is degassed and heated at 100° C. for 2 h. The reaction mixture is cooled, diluted with water and extracted with CH₂Cl₂. The organic fractions are dried and evaporated to give the above titled compound.

e) 3-(5-tert-Butoxy-6-cyano-pyridin-2-yl)-propionic acid ethyl ester

The compound from Example 1(d) and 10% Pd/C in ethanol is treated with H₂ at 60 psi for 2 h. The catalyst is filtered and solvent evaporated to give the above titled compound.

f) 3-(6-Cyano-5-hydroxy-pyridin-2-yl)-propionic Acid Ethyl Ester Hydrochloride Salt

The compound from Example 1 (e) in dioxane is treated with 4N HCl in dioxane at room temperature for 1 h. The reaction is evaporated to give the above titled compound as its HCl salt.

### Example 2

Preparation of 3-[6-Cyano-5-((R)-1-oxiranylmethoxy)-pyridin-2-yl]-propionic Acid Ethyl Ester

A solution of a one-to-one mixture of the compound from Example 1(f) and (2R)-glycidyl 3-nitrobenzenesulfonate in dry acetone is treated with potassium carbonate (3 equivalents) and heated at reflux under argon for 18 h. The reaction is cooled, filtered, and the filtrate is concentrated in vacuo and the residue is purified by flash column chromatography to give the above titled compound.

### Example 3

Preparation of 2-Indan-2-yl-1,1-dimethyl-ethylamine

a) Indan-2-yl-acetic acid Methyl Ester

A solution of indan-2-yl-acetic acid (Lancaster, 20 g, 0.11 mol) in methanol (200 mL) was stirred and cooled to 0-10° C. in an ice bath and treated drop-wise with thionyl chloride (14.8 g, 0.125 mol). The mixture was stirred at RT for 16 h, concentrated in vacuo, and the oily residue was dissolved in ethyl acetate, washed with 2.5 N sodium hydroxide, water, and brine, dried (MgSO₄), and concentrated in vacuo to give the title compound (21 g, 97%) which solidified.

b) 1-Indan-2-yl-2-methyl-propan-2-ol A solution of the compound from Example 3(a) (6.3 g, 33 mmol) in ether (150 mL) was added drop-wise to 1.4 M methyllithium in ether (100 mL, 4.25 eq) stirred in an ice bath. The mixture was allowed to warm to RT, stirred for 2 h, and very carefully quenched by drop-wise addition of saturated aqueous ammonium chloride (150 mL). The aqueous phase was separated and extracted with ether, and the combined ether phase was washed with brine, dried (MgSO₄), and concentrated in vacuo to afford the title compound as an oil which crystallized on standing (˜89%).

c) N-(2-Indan-2-yl-1,1-dimethyl-ethyl)-acetamide

To a mixture of concentrated sulfuric acid (1.7 mL) in acetonitrile (6 mL) stirred in an ice bath for 45 min. a drop-wise solution of the compound from Example 3(b) (3.3 g, 17.3 mmol) in glacial acetic acid (5 mL) was added. The mixture was allowed to warm to RT, stirred for 16 h, poured into ice water, and extracted with ethyl acetate. The combined organic extract was washed with 2.5 N sodium hydroxide, water, and brine, dried (MgSO₄), and concentrated in vacuo to give an oily residue that was triturated with hexane and a few drops of ethyl acetate, seeded, and cooled to afford a solid which was isolated by filtration to afford the title compound as tan solid (1.9 g, 47%). MS(ES) m/e 231.9 [M+H]+. The filtrate was concentrated in vacuo to afford additional title compound as an oil (1.5 g, 37%).

d) 2-Indan-2-yl-1,1- dimethyl-ethylamine

A mixture of the compound from Example 3(c) (6.5 g, 28 mmol) in ethylene glycol (170 mL) was treated with crushed potassium hydroxide pellets (13 g), stirred, and heated to 190° C. for 24 h. The mixture was poured into water and extracted with ethyl acetate. The combined organic phase was washed with brine and extracted with 1 N hydrochloric acid. The combined acidic extract was washed with ethyl acetate, basified with 2.5 N sodium hydroxide, and extracted with ethyl acetate. The combined organic extract was washed with brine, dried (MgSO₄), and concentrated in ivacuo to afford the title compound (3.2 g, 60%). MS(ES) m/e 190.6 [M+H]⁺.

### Example 4

Preparation of 3-{6-Cyano-5-[(R)-2-hydroxy-3-(2-indan-2-yl-1,1-dimethyl-ethylamino)-propoxy]-pyridin-3-yl}-propionic Acid Ethyl Ester Hydrochloride Salt

A one-to-one mixture the compounds from Example 2 and Example 3(d) in absolute ethanol is stirred and heated to reflux for 8 h, cooled, concentrated in vacuo. The residue is dissolved in dichloromethane and acidified with 1.0 N hydrogen chloride in ether to afford the above titled compound.

### Example 5

Preparation of 3-{6-Cyano-5-[(R)-2-hydroxy-3-(2-indan-2-yl-1,1-dimethyl-ethylamino)-propoxy]-pyridin-3-yl}-propionic acid

A solution of the compound from Example 4 in ethanol and water (3:1) is treated with 2.5 N sodium hydroxide (1. 1 equivalent) and stirred for RT under argon overnight. The ethanol is removed in vacuo, and the pH was adjusted to the isoelectric point with 1 N hydrochloric acid while stirring. The precipitated solid is collected by filtration, washed with water and dried in vacuo to afford the title compound.

What is claimed is:

1. A compound according to formula (I) hereinbelow:
or a pharmaceutically acceptable salt thereof. 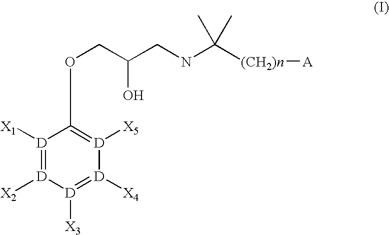
wherein:
A is an aryl or fused aryl, dihydro or tetrahydro fused aryl, heteroaryl or fused heteroaryl, dihydro or tetrahydro fused heteroaryl, unsubstituted or substituted with any substituent being selected from the group consisting of OH, halogen, C₁₋₄alkyl, C₁₋₄alkoxy, C₃₋₆ cycloalkyl, CF₃, OCF₃, CN, and NO₂;
D is C or N with 1-2-N in ring provided that X₁-X₅ are not present when D is N;
X₁ and X₅ are, independently, selected from the group consisting of H, halogen, CN, and NO₂, provided that either X₁ or X₅ is H; further provided that X₁ and X₅ are not present when D is N;
X₂, X₃ and X₄ are selected from the group consisting of H, halogen, O—C₁₋₄ alkyl, and J-K, wherein:
J is a covalent bond, alkylene, O-alkylene or alkenylene; and
K is selected from the group consisting of, CO₂R₅, CONR₄R′₄, OH, NR₄R′₄ and CN and provided X₂, X₃ and X₄ are not present when D is N;
R₄ and R′₄ are independently H, alkyl, aryl or heteroaryl;
R₅ is H, alkyl, alkyl-(O-alkyl)m-O-alkyl, aryl or heteroaryl;
n is an integer from 0 to 4; and,
m is an integer from 1-3.

2. A compound according to claim 1 selected from the group consisting of:
3-{6-Cyano-5-[(R)-2-hydroxy-3-(2-indan-2-yl-1,1-dimethyl-ethylamino)-propoxy]-pyridin-2-yl}-propionic acid ethyl ester;
3-{6-Cyano-5-[(R)-2-hydroxy-3-(2-indan-2-yl-1,1-dimethyl-ethylamino)-propoxy]-pyridin-2-yl}-propionic acid;
3-(6-Cyano-5-{(R)-2-hydroxy-3-[2-(4-methoxy-phenyl)-1,1-dimethyl-ethylamino]-propoxy}-pyridin-2-yl)-propionic acid ethyl ester;
3-(6-Cyano-5-{(R)-2-hydroxy-3-[2-(4-methoxy-phenyl)-1,1-dimethyl-ethylamino]-propoxy}-pyridin-2-yl)-propionic acid;
3-(6-Cyano-5-{(R)-2-hydroxy-3-[4-(2-methoxy-phenyl)-1,1-dimethyl-butylamino]-propoxy}-pyridin-2-yl)-propionic acid ethyl ester; and
3-(6-Cyano-5-{(R)-2-hydroxy-3-[4-(2-methoxy-phenyl)-1, 1-dimethyl-butylamino]-propoxy}-pyridin-2-yl)-propionic acid.

3. A method of antagonizing a calcium receptor, which comprises administering to a subject in need thereof, an effective amount of a compound according to claim 1.

4. A method of treating a disease or disorder characterized by an abnormal bone or mineral homeostasis, which comprises administering to a subject in need of treatment thereof an effective amount of a compound of claim 1.

5. A method according to claim 4 wherein the bone or mineral disease or disorder is selected from the group consisting of osteosarcoma, periodontal disease, fracture healing, osteoarthritis, joint replacement, rheumatoid arthritis, Paget's disease, humoral hypercalcemia, malignancy and osteoporosis.

6. A method according to claim 5 wherein the bone or mineral disease or disorder is osteoporosis.

7. A method according to claim 6 wherein the compound is co-admninistered with an anti-resorptive agent.

8. A method according to claim 7 wherein the anti-resorptive agent is selected from the group consisting of estrogen, 1, 25 (OH)₂ vitamin D3, calcitonin, selective estrogen receptor modulators, vitronectin receptor antagonists, V−H+-ATPase inhibitors, src SH2 antagonists, bisphosphonates and cathepsin K inhibitors.

9. A method of increasing serum parathyroid levels which comprises administering to a subject in need of treatment an effective amount of a compound of claim 1.

10. A method according to claim 9 wherein the compound is co-administered with an anti-resorptive agent.

11. A method according to claim 10 wherein the anti-resorptive agent is selected from the group consisting of: estrogen, 1, 25 (OH)₂ vitamin D3, calcitonin, selective estrogen receptor modulators, vitronectin receptor antagonists, V−H+-ATPase inhibitors, src SH2 antagonists, bisphosphonates and cathepsin K inhibitors.
